(19) United States
(12) Patent Application Publication
(10) Pub. No.: US 2016/0135379 A1
(43) Pub. Date: May 19, 2016

(54) DICOTYLEDON TRANSGENIC METHOD FOR INVADING GROWING POINTS OF SEED SPROUTS OR SEEDLING STEMS MINIMALLY AND FULLY

(71) Applicants: Haibo Wang, Shijiazhuang (CN); Mengyu Lv, Shijiazhuang (CN); Fushuang Dong, Shijiazhuang (CN); Yanmin Zhang, Shijiazhuang (CN); Zhiheng Ren, Shijiazhuang (CN); Fan Yang, Shijiazhuang (CN); Guozhong Sun, Shijiazhuang (CN); Xian Xu, Shijiazhuang (CN); Xueping Shi, Shijiazhuang (CN); Huanhuan Zhang, Shijiazhuang (CN); He Zhao, Shijiazhuang (CN); Jianfang Chai, Shijiazhuang (CN); Yiping Gao, Shijiazhuang (CN); Yongwei Liu, Shijiazhuang (CN); Jinyong Zhu, Shijiazhuang (CN); Qiufen Han, Shijiazhuang (CN); Qiang Zhang, Shijiazhuang (CN); Huijie Ma, Shijiazhuang (CN); Zhanwu Wang, Shijiazhuang (CN); Junfeng Guan, Shijiazhuang (CN)

(72) Inventors: Haibo Wang, Shijiazhuang (CN); Mengyu Lv, Shijiazhuang (CN); Fushuang Dong, Shijiazhuang (CN); Yanmin Zhang, Shijiazhuang (CN); Zhiheng Ren, Shijiazhuang (CN); Fan Yang, Shijiazhuang (CN); Guozhong Sun, Shijiazhuang (CN); Xian Xu, Shijiazhuang (CN); Xueping Shi, Shijiazhuang (CN); Huanhuan Zhang, Shijiazhuang (CN); He Zhao, Shijiazhuang (CN); Jianfang Chai, Shijiazhuang (CN); Yiping Gao, Shijiazhuang (CN); Yongwei Liu, Shijiazhuang (CN); Jinyong Zhu, Shijiazhuang (CN); Qiufen Han, Shijiazhuang (CN); Qiang Zhang, Shijiazhuang (CN); Huijie Ma, Shijiazhuang (CN); Zhanwu Wang, Shijiazhuang (CN); Junfeng Guan, Shijiazhuang (CN)

(21) Appl. No.: 14/422,650

(22) PCT Filed: Sep. 14, 2012
(86) PCT No.: PCT/CN2012/001266

§ 371 Date: Feb. 19, 2015

(30) Foreign Application Priority Data
Aug. 22, 2012 (CN) 201210300379.9

(51) Int. Cl. A01G 1/00

(52) U.S. Cl. CPC A01G 1/001

(57) ABSTRACT

The present invention is a method of shoot apical meristem transformation for dicot plant via sufficient and micro wounding (SMW). The technical process includes: germinate the seeds in Petri dish or in the nutrient matrix which can be transplanted with the seedling; expose the shoot apical meristem by removing one cotyledon away; make sufficient and micro wounding transformation treatment in vivo to the apical meristem by stabbing and brushing for 2-3 times using the SMW brush having 100-5000 bristles which is 4-20 μm in diameter for each one and 0.5-3 mm in exposed length, and dipped with the Agrobacterium tumefaciens containing binary vector harboring exogenous genes; after co-cultivation, develop the treated objects directly to normal plants in the nutrient matrix and then transplant the matrix and seedling together into pot or field; promote the plant to develop more branches, pods, bolls, fruits, and seeds; harvest the seeds of each branch of individual T₀ plants separately; detect and identify the transformation results in T₁ generation from every T₀ plant. The advantages of the invention are independent of tissue culture, unlimited in genotype, no need to graft, unnecessary to carry resistant marker, high survival rate in transplantation and seedlings recovered rapidly, easy to manipulate and large scale to perform, and applicable to all dicot plants which can set seeds. The transformation efficiencies for cotton and soybean using this method are 50% and 76.5%, respectively.

### TECHNICAL FIELD

The present invention is a method of shoot apical meristem transformation for dicot plant via sufficient and micro wounding (SMW), applicable to all dicot plants which can set seeds.

### BACKGROUND ART

Transferring gene via A. tumefaciens is the widely recognized method among a plurality of transformation approaches for plants. It has several advantages including high fertility in transgenic plant, single or low copy number for exogenous gene integration, and long fragment of DNA also can be transferred, etc. However, the conventional transformation via A. tumefaciens is dependent on tissue culture which is limited in genotype, complicated to perform, necessary to carry resistant marker for selection, high somatic variation, low efficiency, and poorly repeatable. For soybean and cotton, as represents for dicot plant, the transformation via A. tumefaciens is only success in the genotypes wherein tissue and cell culture is easily. Thus, the approach is not practicable in application.

The shoot apical meristem of seed or seedling is the original cells which can develop and differentiate to the whole reproduction and the most organs of aerial part. The apical meristem is one of the most ideal objects for transformation, due to its strong ability in regeneration and compensation for development, which can grow to normal plant after one cotyledon and little leaves were removed away and even the cells suffered distinct wounding.

Some reports and patents have mentioned the advantages of transformation using apical meristem as object. However, practical and efficient approach has not been established, due to the poor understanding on characteristics of apical meristem. Such as: the transformation is usually performed in vitro, which is also limited in genotype; the obtained transgenic plant is hard to transplant, and has to be grafted which need long time to recover; and some transformations were conducted without any wounding or sufficient wounding which lead to low efficiency.

Chinese patent, a transformation method for plants with big seeds and the application (NO: 01104428.4), has mentioned the main steps for transformation: (1) germinate the seeds or mature embryos or immature embryos as the transformation objects; (2) remove the coleoptile or cotyledons and little leaves away of young seedlings at appropriate time to expose the stem tip; (3) transform the stem tip with A. tumefaciens to transfer the exogenous genes to the shoot apical meristem cells; (4) conduct resistant screening to select the resistant plants when the plants has developed 3-4 new leaves after transformation; (5) perform the resistant test and molecular identification in progenies of the plants and select the transgenic plants.

Said plants with big seeds comprise soybean and cotton, etc.

Said ‘appropriate time’ is dependent on species. It is the moment that the apical meristem is at highest susceptibility to A. tumefaciens infection.

The main problems of this patent are below:
(1) It is too indistinct, which is lack of qualification of necessary conditions, such as whether and how to make wounding to apical meristem is not clear. It is an uncertain method and hard to guarantee to obtain the transgenic plant.
(2) It is unreasonable to spray the selection chemicals when the plants have developed 3-4 new leaves after transformation, which would easily eliminate the chimera, in which the reproductive organs have been transformed while other organs has not. On the other hand, the resistant plant selected may be only with transformed vegetative organs but the reproductive organs have not been transformed.

And problems exist in other patents and reports for transformation of apical meristem, most of all need in vitro culture which is limited in genotypes, and usually conduct in improper screening strategies.

### SUMMARY OF THE INVENTION

The purpose of this invention is to establish a novel method for shoot apical meristem transformation of dicot plant via sufficient and micro wounding (SMW), which is independent of tissue culture, conducted in vivo, no need to graft, easy to transplant and seedling recovered rapidly, unnecessary to carry resistant marker, high efficient, easy to perform in large scale, stable, practical, and low cost.

Technical scheme for this invention is below:

A method of shoot apical meristem transformation for dicot plant via sufficient and micro wounding, which characterized in that:

(1) Basic Preparation

Place two layers of absorbent tissue in a Petri dish and autoclave them. Make the nutrient matrix, a paper column diameter in 2-5 cm full with vermiculite, which can be transplanted with the seedlings together, and place the matrix longitudinally in a suitable size plastic box.

(2) Preparation of In Vivo Objects and Infection Solution

Select healthy and completed seeds and sterilize them routinely, and then rinse them in sterilized water for 3-5 times. For the plants with folded cotyledons which hardly to be separated, sow the seeds in said nutrient matrix 0.5-1.0 cm deep, and water them from inner wall of the plastic box until the top of the vermiculite is wet. Thereafter, cover the box with lid for 3 days. For the plants with cotyledons which easily to be separated, place the seeds in the Petri dish. Drip appropriate quantities of sterilized water just to make the seeds fully imbibed, and then germinate them for 2-3 days until the roots grow to more than 0.4 cm in length. Said in vivo objects for transformation are the shoot apical meristems of germinated seeds and seedlings.

Growing Condition: At 25° C. In Dark.

Said plant with folded cotyledons which hardly to be separated comprises cotton. Said plants with cotyledons which easily to be separated comprise soybean, mung bean, and cucumber.

Screen single colony of A. tumefaciens containing binary vector harboring exogenous genes, and inoculate it into LB medium containing 50 mg/L kanamycin and 40 mg/L rifampicin and grow to OD₆₀₀=0.5-0.6 at 28° C. on shaker with 220 rpm in dark. Prepare the A. tumefaciens infection solution by centrifugating the culture at 4000 rpm for 5 min and re-suspend it in base buffer of ⅕-¼ volume as the original.

Said base buffer contains 1/10 MS medium with 100 μM AS, 100 mg/L F68, 400 mg/L MES, 30 g/L glucose and 68 g/L sucrose, pH 5.6.

(3) Expose the Apical Meristem and Transform it Using SMW Brush

Remove one cotyledon away to expose the shoot apical meristem. Stab and brush the apical meristem for 2-3 times using the SMW brush dipped with the A. tumefaciens infection solution.

(4) Co-Cultivation

After transformation treatment, for the plants with folded cotyledons which hardly to be separated, cover the box containing said nutrient matrix with lid; for the plants with cotyledons which easily to be separated, place the treated seeds with the exposed side up in the Petri dish containing two layers of absorbent tissue which has been wetted with sterilized water, and then cover the lid.

Co-Cultivation Condition: At 25° C. In Dark for 3 Days.

(5) Develop to Seedlings and Transplantation

After co-cultivation, for the plants in said nutrient matrix, open the lid of the plastic box and grow the plants under light until the first leaf is expanded; for the plants with cotyledons which easily to be separated and growing in Petri dish, treat them under light for one day, and then transplant each one in said nutrient matrix with root down and grow them under light also until the first leaf is expanded.

Growing Condition: At 25° C. With a 12-h Photoperiod.

After growing to seedlings, transplant them with the said nutrient matrix into the environmentally controlled greenhouse.

(6) Seedling and Plant Management

Promote the seedlings to healthy plants and develop more branches, pods, bolls, fruits, and seeds with suitable light, temperature, water, and nutrition management.

(7) Identification

To avoid false results, do not perform the detection, selection and identification in T₀ plants. Harvest the seeds of each branch separately of each individual T₀ plant. After germination and growing to seedlings, perform the molecular detection and identification in T₁ generation. For the plants which have been transformed with exogenous vector harboring resistant gene, carry out PCR identification with resistant plants after resistance screening; for the plants transformed without any resistant gene, carry out PCR identification directly. Perform Southern blot analysis among PCR-positive plants.

Said SMW brush, the bristles are made of stainless steel fibers, glass fibers or carbon silicon fibers in micron-grade. One bristle is 4-20 nm in diameter and 0.5-3 mm in exposed length, and each brush contains 100-5000 bristles.

Said SMW brush in which bristle is 8-18 μm in diameter, the bristle is 1-2 mm in exposed length, and each brush contains 100-2000 of bristles.

Said “stab and brush” is mean not only to stab but also to brush on the apical meristem. Said “stab” is to prick the apical meristem vertically with the SMW brush dipped with the A. tumefaciens infection solution to transfer the exogenous genes; and said “brush” is to comb the whole apical meristem with the SMW brush dipped with the A. tumefaciens infection solution to transfer the exogenous genes.

Technical Principle of the Invention is Below:

The applicants find that: (1) for dicot plants, such as cotton and soybean, when their cotyledons can be opened after the seeds have germinated, one cotyledon is removed away to expose the apical meristem, they also can develop to normal plants. For the problem that roots of the seedlings are usually to be hurt during transplantation, which could affect the survival rate and later development, the applicants set up a column ‘nutrient matrix’, which can develop the cotyledons of dicot plants to open as early as possible, and also can be transplanted with the seedlings together to keep the roots avoid damage. With this technique, in vivo transformation for shoot apical meristem can be conducted as early as possible, and the transplantation survival can be guaranteed. (2) In order to obtain good transformation result, sufficient and micro wounding to the tender apical meristem is necessary. Thus, the applicants invent a novel instrument for plant transformation, which can make sufficient and micro wounding to apical meristem. So it is called sufficient and micro wounding brush, abbreviated as SMW brush. The brush contains 100-5000 bristles (4-20 μm in diameter for each) which are made of stainless steel fibers, glass fibers or carbon silicon fibers. Good transformation can be obtained using this kind of brush dipped with A. tumefaciens infection solution. (3) Results can be improved by properly controlling the water potential of the seedling after transformation, in order to avoid the cells burst or wilting and to promote the A. tumefaciens close to the meristem cells. (4) After being treated with SMW brush, most of the shoot meristems can normally develop to blossom, pod or ball or fruit, and set seeds, and the transformation save time with higher efficiency compared to other methods. (5) The seeds from each pod or ball or fruit, branch, and plant are harvested separately, and then to be germinated to seedlings. Molecular identification is performed in T₁ generation. This strategy no longer needs resistant screen and special selective mark. The results can accurately show the transformation pattern of the whole plant. (6) This method is also suitable to mung bean and cucumber, etc.

Based on these recognitions, the special evaluation index is established for this transformation technique:

The damage rate and the normal seedling rate: after stabbing and brushing, some of the shoot apical meristems are usually damaged and cannot develop normally. The damage rate is the percentage of the treated meristems which cannot develop normally. Conversely, normal seeding rate is the percentage of the treated meristems which can develop normally.

$$The damage rate = \frac{\begin{matrix}number of treated meristems - \\ number of normal seedlings\end{matrix}}{number of treated meristems} \times 100 \%$$

$$The normal seedling rate = \frac{number of normal seedlings}{number of treated meristems} \times 100 \%$$

The transformation rate is the percentage of T₀ plants which set positive seeds proved in T₁ generation.

$$The transformation rate = \frac{number of {T}_{0} plants positive seeds}{number of {T}_{0} plants setting seeds} \times 100 \%$$

The transformation degree is the percentage of the positive seeds for each individual T₀ plant. It can be calculated from every ball or pod or fruit and branch to form a transformation pattern of a whole plant. This index reflects the transformation degree and the transformed status for an apical meristem.

$$The transformation degree = \frac{number of positive seeds of a {T}_{0} plant}{number of all seeds of the {T}_{0} plant} \times 100 \%$$

The advantages of the invention make A. tumefaciens-mediated transformation of dicot plants are no longer to need tissue culture and carry resistant marker, unnecessary to transform in vitro and graft, not difficult to transplant and recover the seedlings. It is easy to perform in large scale, high efficiency, stable, practical, low cost, and applicable to all dicot plants which can set seeds.

### BRIEF DESCRIPTION OF THE FIGURES

FIG. 1 The structure of the SMW brush. A: The SMW brush with bristles made of stainless steel fibers; B: The SMW brush with bristles made of glass fibers; C: The SMW brush with bristles made of carbon silicon fibers; a: The SMW brush has 4000 bristles which are 8 μm in diameter for each and made of stainless steel fibers; b: The SMW brush has 200 bristles which are 4 μm in diameter for each and made of stainless steel fibers; c: The SMW brush has 200 bristles which are 16 μm in diameter for each and made of stainless steel fibers.

FIG. 2 The longitudinal structure of the apical meristem of dicot plants through micro observation. a: The longitudinal structure of the apical meristem of cotton through micro observation; b: The longitudinal structure of the apical meristem of soybean through micro observation.

FIG. 3 Album of sufficient and micro wounding transformation for shoot apical meristem of cotton, a typical dicot plant. a: The seedlings to make objects for transformation; b: Microscope view for apical meristem (framed by the circle); c: The head of SMW brush; d: Transformation via the SMW brush; e: Transformation treated meristems of the seedlings with one cotyledon; f: T₀ plants; g: T₁ seedling; h: Some results of PCR.

FIG. 4 Album of all kinds of the transforms and their progenies via sufficient and micro wounding transformation. a: The environmentally controlled greenhouse for transgenic plant; b: T₂ plants of transgenic cotton; c: T₀ plants of transgenic soybean; d: T₂ plants of transgenic soybean; e: T₀ plants of transgenic mung bean; f: T₀ plants of transgenic cucumber.

FIG. 5 PCR results of NPT-II gene in T₁ resistant plants of transgenic cotton.

FIG. 6 Southern blot analysis for transgenic cotton genomes. Lane ‘CK⁺’ is the vector control, lane ‘Ck⁻’ is the negative control, and lane 1 to lane 11 are the detected samples.

FIG. 7 PCR results of BAR gene in T₁ plants of transgenic soybean.

FIG. 8 Southern blot analysis for T₂ transgenic soybean genomes. Lane ‘M’ is the marker, lane 1 to lane 8 are the detected samples, lane 9 is the vector control, and lane 10 is the PCR product control.

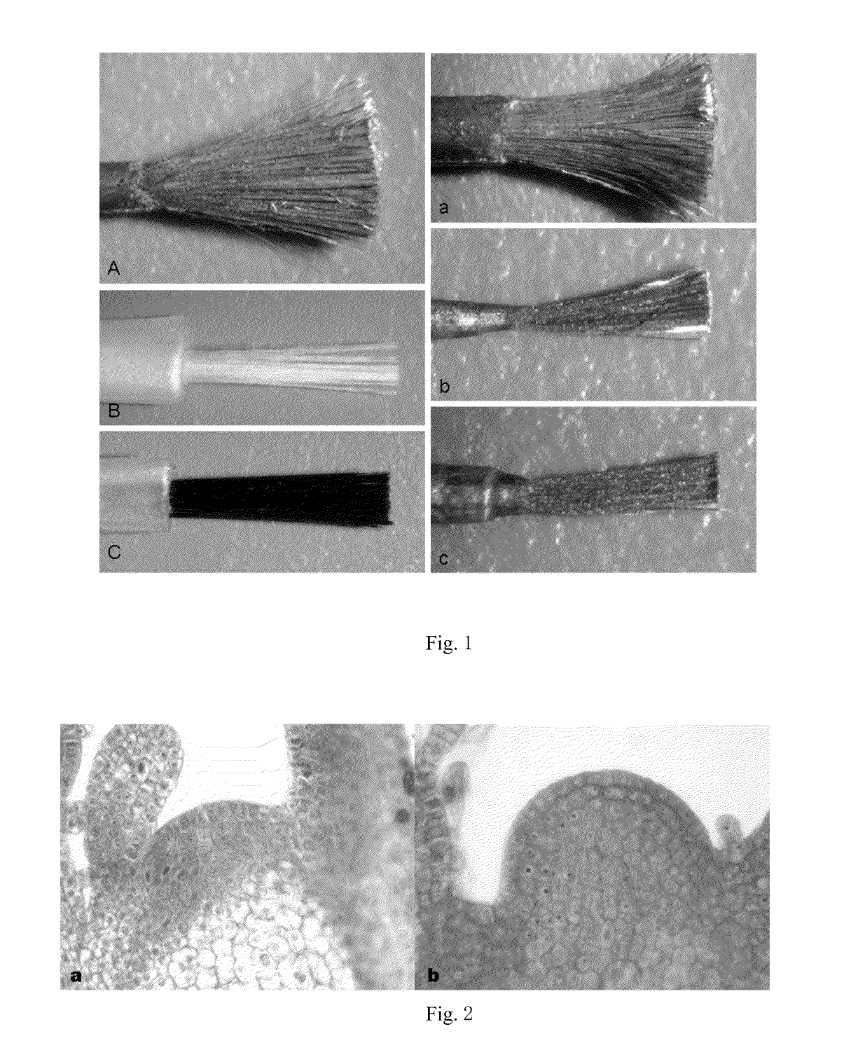

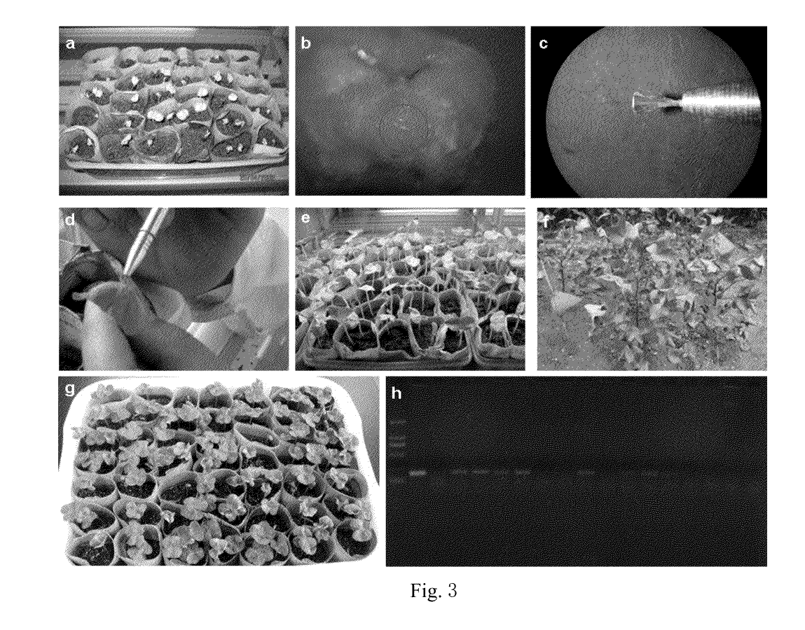

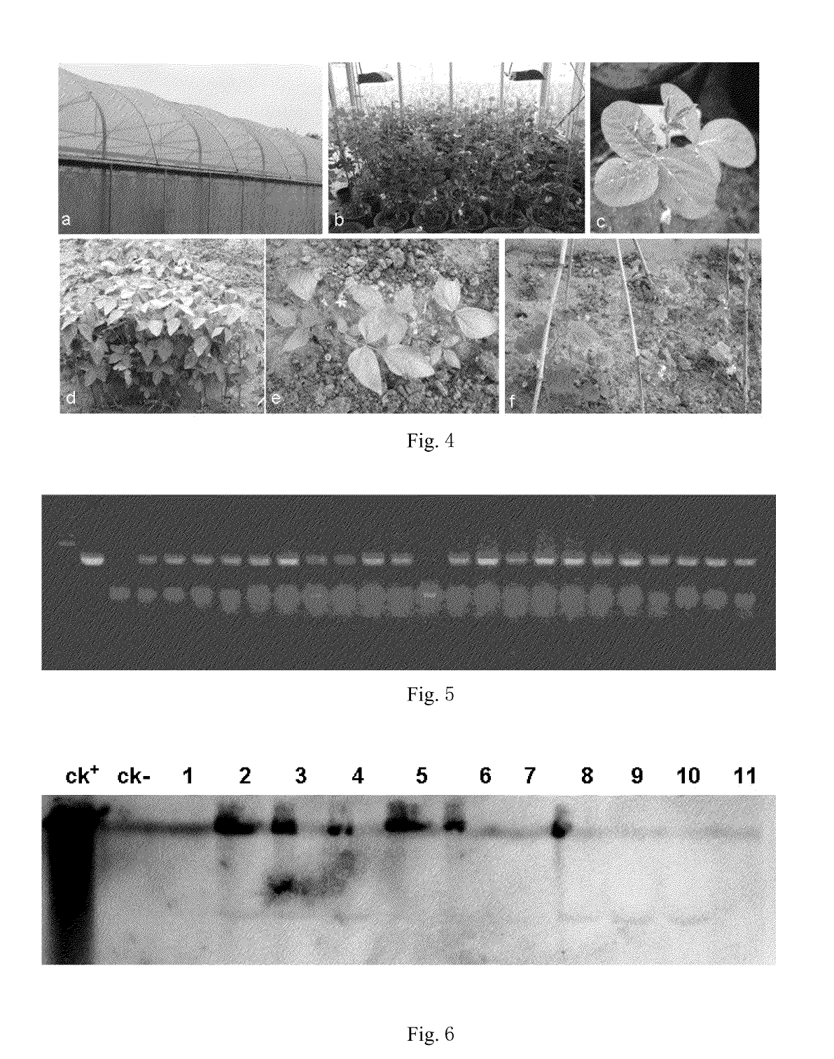

### DETAILED DESCRIPTION OF THE PREFERRED EMBODIMENT

### Embodiment 1

### The Transformation for Shoot Apical Meristem of Cotton Using the SMW Brush

1. Materials and Methods

Cotton Cultivar: Jimian 27.

A. tumefaciens strain: C58C1.

The Exogenous Genes: Gus Gene and Npt-II Gene, Constructed in Vector pCAMBIA2201.

Single colony of A. tumefaciens was screened and inoculated into 100 mL of LB medium containing 50 mg/L kanamycin and 40 mg/L rifampicin, and grew to OD₆₀₀=0.5 at 28° C. on shaker with 220 rpm. The A. tumefaciens infection solution was obtained by centrifugating the culture at 4000 rpm for 5 min and re-suspending base buffer (⅕ volume of the original) containing 1/10 MS medium complemented with 100 μM AS, 100 mg/L F68, 400 mg/L MES, 30 g/L glucose and 68 g/L sucrose, pH 5.6.

The nutrient matrix was made 2-5 cm in diameter of a column by rolling paper to a tube and full with vermiculite, which could be transplanted with the seedling together. They were placed longitudinally in a plastic box. 40 healthy and complete seeds were soaked in water for 7 hours and sowed 1 cm deep in said nutrient matrix, and then the vermiculite was fully watered and the whole box was covered with lid. The box was kept at 25° C. in dark for 3 days until the cotyledons were open. Then one cotyledon was removed away to expose the shoot apical meristem. The exposed meristem was stabbed and brushed for 2-3 times using SMW brush (5000 bristles which are 8 μm in diameter for each, 2 mm in exposed length) dipped with the A. tumefaciens infection solution. The box was covered with lid and placed at 25° C. in dark for 3 days. Then the plants grew in the box without lid. When one leaf expanded under a 12-h photoperiod, they were transplanted into the environmentally controlled greenhouse with said nutrient matrix.

The cotton balls were harvested and the naked seeds were made and sowed separately according to each T₀ plant. 10 g/L kanamycin was smeared to the leaves of the seedlings to screen resistant plants. Total genomic DNA was extracted individually from some leaves of every kanamycin resistant plant. PCR was conducted with npt-II fragment primers: forward 5′-TGT TCC GGC TGT CAG CGC AG-3′ and reverse 5′-TCG GCA AGC AGG CAT CGC CA-3′. Based on the results of PCR amplification, the transformation rate and transformation degree were calculated. Second PCR was performed in inbred progenies from first PCR positive plants, and then Southern blot was conducted in the second PCR positive plants.

2. Results

31 out of 40 seeds germinated normally and grew to seedlings. They were used for transformation to the shoot apical meristem and 14 of them developed normally to plants (normal seedling rate was 45.2%). 605 seeds were harvested from the plants and due to the low temperature of greenhouse, about ⅔ of them were wizened. The full seeds were screened and germinated separately according to the individual T₀ plant: 213 of them grew to seedlings and they were from 12 T₀ plants; 2 of them didn't grow to seedling due to their low maturity. Based on kana-resistant selection, PCR analysis was carried out. 44 of the seedlings were positive and they were from 6 T₀ plants (FIG. 5). The transformation rate was 50% (6/12×100%) and the transformation degree for each T₀ plant was 2.3%, 11.1%, 14.3%, 33.3%, 57.1%, and 53.8%, respectively. Southern blot analysis was performed in PCR-positive plants of T₂ generation, which indicated that the exogenous npt-II gene has integrated in cotton genome (FIG. 6).

### Embodiment 2

### The Transformation for Apical Meristem of Soybean Using the SMW Brush

1. Materials and Methods

Soybean Cultivar: Jidou 16.

A. tumefaciens strain: EHA105.

The Exogenous Genes: Bar Gene, Pta Gene and Bt Gene, Constructed in Vector pCAMBIA3300.

Single colony of A. tumefaciens was screened and inoculated into 100 mL of LB medium containing 50 mg/L kanamycin and 40 mg/L rifampicin, and grew to OD₆₀₀=0.6 at 28° C. on shaker with 220 rpm. The A. tumefaciens infection solution was obtained by centrifugating the culture at 4000 rpm for 5 min and re-suspended in the base buffer (¼ volume of the original) containing 1/10 MS medium complemented with 100 μM AS, 100 mg/L F68, 400 mg/L MES, 30 g/L glucose and 68 g/L sucrose, pH 5.6.

Two layers of absorbent tissue were placed in each Petri dish (Φ 9 cm), and they were autoclaved for the experiment. 21 healthy soybean seeds were selected and germinated in the Petri dish containing 10 mL of sterilized water at 25° C. in dark for 3 days. Then one cotyledon was removed away to expose the apical meristem. The exposed meristem was stabbed and brushed for 2-3 times using SMW brush (2000 bristles which are 20 μm in diameter for each, 3 mm in exposed length) dipped with the A. tumefaciens infection solution. Thereafter, the treated objects were placed in the Petri dish (the tissue soaked with sterilized water) with the exposed side up. The Petri dish was covered with lid and placed at 25° C. in dark for 3 days. The seedlings with single cotyledon were cultured for one day under light in the Petri dish and then were planted in said nutrient matrix (fully watered) with root down. When one leaf of the seedlings was fully expanded, they were transplanted with the said nutrient matrix into the environmentally controlled greenhouse.

The seeds were harvested and sowed separately according to each T₀ plant. When the seeds grew to seedlings, total genomic DNA was extracted individually from some leaves of every plant. PCR was conducted with bar fragment primers: forward 5′-ATG AGC CCA GAA CGA CGC C-3′ and reverse 5′-TCA GAT CTC GGT GAC GGG CA-3′. The transformation rate and transformation degree were calculated according to the PCR results. The inbred progeny from the PCR-positive plant was identified again by PCR. Southern blot analysis was conducted in the second PCR positive plants (performed by Beijing Meilaibo Medical Technology Co. Ltd.)

2. Results

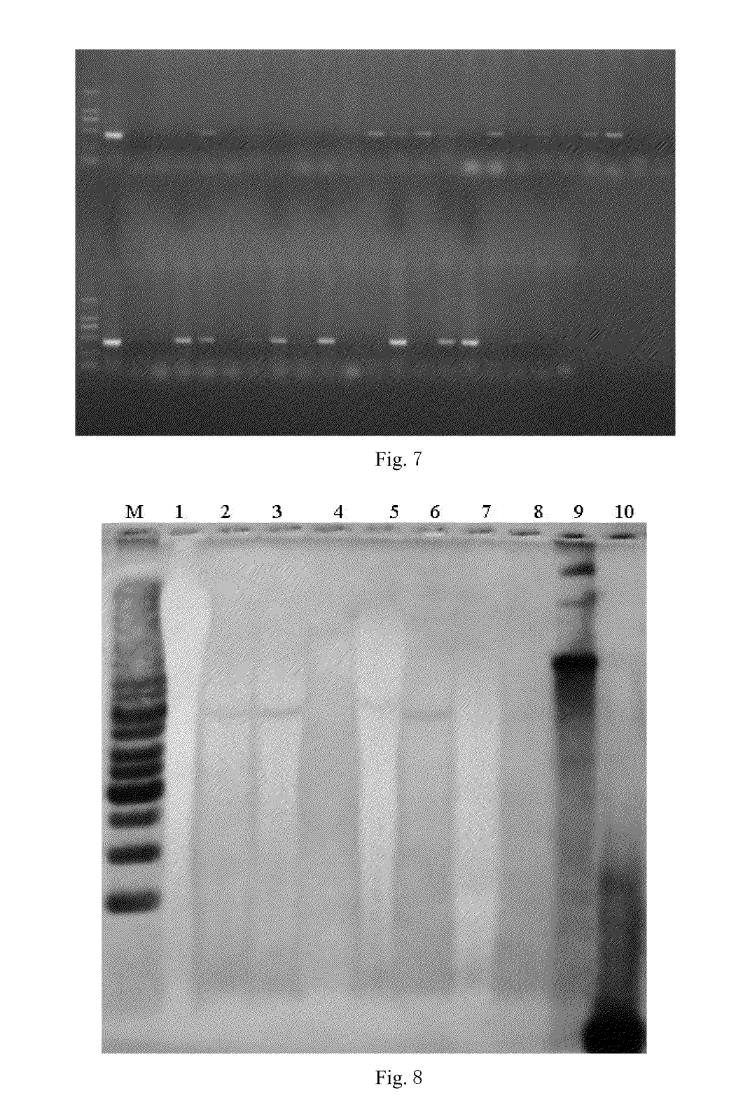

19 out of 21 seeds germinated normally and were used for transformation. 17 of treated objects developed normally to plants and 66 seeds were harvested. They were sowed separately according to the individual T₀ plant and 61 of them grew to seedlings. Based on the PCR results, 27 of the seedlings were positive and they were from 13 T₀ plants (FIG. 7). The transformation rate was 76.5% ( 13/17×100%). Although the seeds were few, the transformation degree was high, which reached to 100% in 2 T₀ plants, 66.7% in 3 T₀ plants, 50% in 4 T₀ plants, and 33.3-46.2% in 4 T₀ plants, respectively. Southern blot analysis was performed in some PCR-positive plants, and indicated that the exogenous gene was single copy integration in soybean genome (FIG. 8).

### Embodiment 3

### The Transformation for Apical Meristem of Different Genotypes of Soybean Using the SMW Brush

1. Materials and Methods

Soybean Cultivars: Jidou 12, Jidou 17.

A. tumefaciens strain: EHA105.

The Exogenous Genes: Bar Gene, Pta Gene and Bt Gene, Constructed in Vector pCAMBIA3300.

Single colony of A. tumefaciens was screened and inoculated into 50 mL of LB medium containing 50 mg/L kanamycin and 40 mg/L rifampicin, and grew to OD₆₀₀=0.6 at 28° C. on shaker with 220 rpm. The A. tumefaciens infection solution was obtained by centrifugating the culture at 4000 rpm for 5 min and re-suspended in the base buffer (⅕ volume of the original) containing 1/10 MS medium complemented with 100 μM AS, 100 mg/L F68, 400 mg/L MES, 30 g/L glucose and 68 g/L sucrose, pH 5.6.

Two layers of absorbent tissue were placed in each Petri dish (Φ 9 cm), and they were autoclaved for the experiment. 30 healthy seeds of each soybean cultivar were selected and germinated in the Petri dish containing 10 mL of sterilized water at 25° C. in dark for 2 days. Then one cotyledon was removed away to expose the apical meristem. The exposed meristem was stabbed and brushed for 2-3 times using SMW brush (2000 bristles which are 18 μm in diameter for each, 3 mm in exposed length) dipped with the A. tumefaciens infection solution. Thereafter, the treated objects were placed in the Petri dish (the tissue soaked with sterilized water) with exposed side up. The Petri dish was covered with lid and placed at 25° C. in dark for 3 days. The seedlings with single cotyledon were cultured about one day under light in the Petri dish until the root grew to more than 0.4 cm in length, and then they were planted in said nutrient matrix (fully watered) with root down. When one leaf of the seedlings was fully expanded, they were transplanted with the said nutrient matrix into the environmentally controlled greenhouse.

The seeds were harvested and sowed separately according to each T₀ plant. When the seeds grew to seedlings, total genomic DNA was extracted individually from some leaves of every plant. PCR was conducted with bar fragment primers: forward 5′-ATG AGC CCA GAA CGA CGC C-3′ and reverse 5′-TCA GAT CTC GGT GAC GGG CA-3′. The transformation rate was calculated according to the PCR results.

2. Results

Two genotypes of soybean were all successfully transformed. The details were showed below:

Jidou 12: All 30 seeds germinated normally and 28 of them were used for transformation. 23 of treated objects developed to plants and 44 seeds were harvested. They were sowed separately according to the individual T₀ plant and 42 of them grew to seedlings. Based on the PCR results, 9 of the seedlings were positive and they were from 8 T₀ plants. The transformation rate was 34.8% ( 8/23×100%).

Jidou 17: All 30 seeds germinated normally and 29 of them were used for transformation. 27 of treated objects developed to plants and 78 seeds were harvested. They were sowed separately according to the individual T₀ plant and all of them grew to seedlings. Based on the PCR results, 9 of the seedlings were positive and they were from 8 T₀ plants. The transformation rate was 29.6% ( 8/27×100%).

### Embodiment 4

### The Transformation for Apical Meristem of Mung Bean Using the SMW Brush

1. Materials and Methods

Mung Bean Cultivar: Jilv 7.

A. tumefaciens strain: EHA105.

The Exogenous Genes: Bar Gene, Pta Gene and Bt Gene, Constructed in Vector pCAMBIA3300.

Single colony of A. tumefaciens was screened and inoculated into 50 mL of LB medium containing 50 mg/L kanamycin and 40 mg/L rifampicin, and grew to OD₆₀₀=0.6 at 28° C. on shaker with 220 rpm. The A. tumefaciens infection solution was obtained by centrifugating the culture at 4000 rpm for 5 min and re-suspended in the base buffer (¼ volume of the original) containing 1/10 MS medium complemented with 100 μM AS, 100 mg/L F68, 400 mg/L MES, 30 g/L glucose and 68 g/L sucrose, pH 5.6.

Two layers of absorbent tissue were placed in each Petri dish (Φ 9 cm), and they were autoclaved for the experiment. 20 healthy mung bean seeds were selected and germinated in the Petri dish containing 5 mL of sterilized water at 25° C. in dark for one day. Then one cotyledon was removed away to expose the shoot apical meristem. The exposed meristem was stabbed and brushed for 2-3 times using SMW brush (100 bristles which are 10 μm in diameter for each, 1 mm in exposed length) dipped with the A. tumefaciens infection solution. Thereafter, the treated objects were placed in the Petri dish (the tissue soaked with sterilized water) with the exposed side up. The Petri dish was covered with lid and placed at 25° C. in dark for 3 days. The seedlings with single cotyledon were cultured for one day under light in the Petri dish and then were planted in said nutrient matrix (fully watered) with root down. When one leaf of the seedlings was fully expanded, they were transplanted with the said nutrient matrix into the environmentally controlled greenhouse.

The seeds were harvested and sowed separately according to each T₀ plant. When the seeds grew to seedlings, total genomic DNA was extracted individually from some leaves of every plant. PCR was conducted with bar fragment primers: forward 5′-ATG AGC CCA GAA CGA CGC C-3′ and reverse 5′-TCA GAT CTC GGT GAC GGG CA-3′. The transformation rate was calculated according to the PCR results.

2. Results

All 20 seeds germinated normally and 15 of them were used for transformation. 11 of treated objects grew to seedlings. However, only 10 of them survived after transplantation and each one produced 2-3 pods. The seeds from the top pod (3-6 seeds) were sowed separately according to the individual T₀ plant and PCR identification was conducted for every seedling. Positive results indicated that 7 T₀ plants were transformed successfully. The transformation rate was 70% ( 7/10×100%).

### Embodiment 5

### The Transformation for Apical Meristem of Cucumber Using the SMW Brush

1. Materials and Methods

Cucumber Cultivar: Oulang-Km567.

A. tumefaciens strain: EHA105.

The Exogenous Genes: Bar Gene, Pta Gene and Bt Gene, Constructed in Vector pCAMBIA3300.

Single colony of A. tumefaciens was screened and inoculated into 50 mL of LB medium containing 50 mg/L kanamycin and 40 mg/L rifampicin, and grew to OD₆₀₀=0.6 at 28° C. on shaker with 220 rpm. The A. tumefaciens infection solution was obtained by centrifugating the culture at 4000 rpm for 5 min andre-suspended in the base buffer (¼ volume of the original) containing 1/10 MS medium complemented with 100 μM AS, 100 mg/L F68, 400 mg/L MES, 30 g/L glucose and 68 g/L sucrose, pH 5.6.

Two layers of absorbent tissue were placed in each Petri dish (Φ 9 cm), and they were autoclaved for the experiment. 30 healthy cucumber seeds were selected and germinated in the Petri dish containing 4 mL of sterilized water at 25° C. in dark for 2 days. Then one cotyledon was removed away to expose the sprout apical meristem. The exposed meristem was stabbed and brushed for 2-3 times using SMW brush (90 bristles which are 4 μm in diameter for each, 0.5 mm in exposed length) dipped with the A. tumefaciens infection solution. Thereafter, the treated objects were placed in the Petri dish (the tissue soaked with sterilized water) with the exposed side up. The Petri dish was covered with lid and placed at 25° C. in dark for 3 days. The seedlings with single cotyledon were cultured for one day under light in the Petri dish and then were planted in said nutrient matrix (fully watered) with root down. When one leaf of the seedlings was fully expanded, they were transplanted with the said nutrient matrix into the environmentally controlled greenhouse.

The seeds were harvested and sowed separately according to each T₀ plant. When the seeds grew to seedlings, total genomic DNA was extracted individually from some leaves of every plant. PCR was conducted with bar fragment primers: forward 5′-ATG AGC CCA GAA CGA CGC C-3′ and reverse 5′-TCA GAT CTC GGT GAC GGG CA-3′. The transformation rate was calculated according to the PCR results.

2. Results

26 out of 30 seeds germinated normally and 19 of them were used for transformation. 14 of treated objects developed to seedlings. After being transplanted to greenhouse, 9 of them survived. Due to the abnormal temperature in summer, 5 of them were hurt by heat stress and 4 of them developed normally. The seeds from the first fruit of the normal plants were sowed separately according to the individual T₀ plant and PCR identification was conducted for every plant. Positive results indicated that 3 T₀ plants were transformed successfully. The transformation rate was 75.0% (¾×100%).

1. A method of shoot apical meristem transformation for dicot plant via sufficient and micro wounding (SMW), comprising the steps of:
(1) Basic Preparation
Placing two layers of absorbent tissue in each Petri dish and autoclaving the absorbent tissue; filling a paper column 2-5 cm in diameter with vermiculite to make a nutrient matrix, which can be transplanted together with seedlings, and placing the nutrient matrix longitudinally in a plastic box;
(2) Preparation of In Vivo Objects and Infection Solution
Selecting healthy and non-damaged seeds and sterilizing them routinely, and then rinsing them in sterilized water for 3-5 times; for the plants with folded cotyledons which hardly to be separated, sowing the seeds in said nutrient matrix 0.5-1.0 cm deep, and watering them from inner wall of the plastic box until the top of the vermiculite is wet, thereafter, covering the box with lid for 3 days; for the plants with cotyledons which easily to be separated, placing the seeds in the Petri dish; dripping appropriate quantities of sterilized water to the seeds fully imbibed, and then germinating the seeds for 2-3 days until the roots grow to more than 0.4 cm in length; said in vivo objects for transformation are the shoot apical meristems of the germinated seeds or seedlings;
Growing condition: at 25° C. in dark;
Said plant with folded cotyledons which hardly to be separated comprises cotton; said plants with cotyledons which easily to be separated comprise soybean, mung bean, and cucumber;
Screening single colony of A. tumefaciens containing binary vector harboring exogenous genes, and inoculating it into LB medium containing 50 mg/L kanamycin and 40 mg/L rifampicin and growing to OD₆₀₀=0.5-0.6 at 28° C. on shaker with 220 rpm in dark; obtaining the A. tumefaciens infection solution by centrifugating the culture at 4000 rpm for 5 min and re-suspend it in base buffer of ⅕-¼ volume as the original;
Said base buffer contains 1/10 MS medium with 100 μM AS, 100 mg/L F68, 400 mg/L IVIES, 30 g/L glucose and 68 g/L sucrose, pH 5.6;
(3) Expose the Apical Meristem and Transform it Using SMW Brush
Removing one cotyledon away to expose the shoot apical meristem; stabbing and brushing the apical meristem for 2-3 times using SMW brush dipped with the A. tumefaciens infection solution;
(4) Co-Cultivation
After transformation treatment, for the plants with folded cotyledons which hardly to be separated, covering the plastic box containing said nutrient matrix with lid; for the plants with cotyledons which easily to be separated, placing the treated objects with the exposed side up in the Petri dish containing two layers of absorbent tissue which has been wetted with sterilized water, and then cover the lid;
Co-cultivation condition: at 25° C. in dark for 3 days;
(5) Develop to Seedlings and Transplantation
After co-cultivation, for the plants in said nutrient matrix, opening the lid of the plastic box and growing the plants under light until the first leaf is expanded; for the plants with cotyledons which easily to be separated and growing in Petri dish, treating them under light for one day, and then transplanting each one in said nutrient matrix with root down and growing them under light also until the first leaf is expanded;
Growing condition: at 25° C. with a 12-h photoperiod;
After growing to seedlings, transplanting them with the said nutrient matrix into the environmentally controlled greenhouse;
(6) Seedling and Plant Management
Promoting the seedlings to healthy plants to develop more branches, pods, bolls, fruits, and seeds with suitable light, temperature, water, and nutrition management.
(7) Identification
To avoid false results, do not perform the detection, selection and identification in T₀ plants; harvesting the seeds of each branch separately of each individual T₀ plant; after germination and growing to seedlings, performing the molecular detection and identification in T₁ generation; for the plants which have been transformed with exogenous vector harboring resistant gene, carrying out PCR identification with the resistant plants after resistance screening; for the plants transformed without any resistant gene, carrying out PCR identification directly; performing Southern blot analysis among PCR-positive plants;
wherein the bristles of said SMW brush are made of stainless steel fibers, glass fibers or carbon silicon fibers in micron-grade, one bristle is 4-20 μm in diameter and 0.5-3 mm in exposed length, and each brush contains 100-5000 bristles.

2. (canceled)

3. The method of claim 1, wherein said SMW brush in which bristle is 8-18 μm in diameter, the bristle is 1-2 mm in exposed length, and each brush contains 100-2000 of bristles.

4. The method of claim 1, wherein said “stabbing and brushing” comprises stabbing and brushing on the apical meristem, said “stabbing” is to prick the apical meristem vertically with the SMW brush dipped with the A. tumefaciens infection solution to transfer the exogenous genes; and said “brushing” is to comb the whole apical meristem with the SMW brush dipped with the A. tumefaciens infection solution to transfer the exogenous genes.


SEQUENCE LISTING (4 sequences)

SEQ ID NO: 1 - DNA, 20 bp, Artificial Sequence
  Synthetically generated
tgttccggctgtcagcgcag

SEQ ID NO: 2 - DNA, 20 bp, Artificial Sequence
  Synthetically generated
tcggcaagcaggcatcgcca

SEQ ID NO: 3 - DNA, 19 bp, Artificial Sequence
  Synthetically generated
atgagcccagaacgacgcc

SEQ ID NO: 4 - DNA, 20 bp, Artificial Sequence
  Synthetically generated
tcagatctcggtgacgggca